(19) United States
(12) Patent Application Publication
(10) Pub. No.: US 2002/0193285 A1
(43) Pub. Date: Dec. 19, 2002

(54) Neuroprotectants formulations and methods

(76) Inventors: David P. Hesson, Malvern, PA (US); Glen D. Frazer, Wynnewood, PA (US); Douglas Ross, North wales, PA (US)

Correspondence Address: ALLEN BLOOM, C/O DECHERT, PRINCETON PIKE CORPORATION CENTER, P.O. BOX 5218, PRINCETON, NJ, 08543-5218, US

(21) Appl. No.: 10/090,441

(22) Filed: Mar. 4, 2002

Related U.S. Application Data
(60) Provisional application No. 60/331,360, filed on Mar. 2, 2001.

(51) Int. Cl. A61K 31/00

(52) U.S. Cl. 514/1

(57) ABSTRACT

Provided is a method of treating in an animal that has suffered damage to cerebrospinal tissue or that has an indication creating a risk of damage to cerebrospinal tissue, the method comprising: a. injecting a physiologically acceptable cerebrospinal perfusion fluid into a first catheter into the cerebrospinal pathway, which cerebrospinal perfusion fluid has a neuroprotecting effective amount of a neuroprotectant; b. withdrawing fluid at a second catheter into the cerebrospinal pathway to create a flow and flow pathway between the first and second catheters; and c. maintaining the flow for a period of time adapted to perfuse an affected tissue.

This application claims the priority of U.S. Provisional Application No. 60/ ______ (pursuant to a petition converting U.S. patent application Ser. No. 09/798,880 to a provisional), which application was filed Mar. 2, 2001.

This invention relates to neuroprotectant formulations or compositions useful for treating neurologic diseases and conditions that cause death or damage to neurological tissue, such as Alzheimer's disease, multiple sclerosis, amyotrophic lateral sclerosis, stroke, traumatic brain injury (TBI), brain hemorrhage, spinal cord traumatic injury, ischemia caused by surgical intervention or central nervous system ischemic or chemical injury.

The cerebrospinal fluid (CSF) pathway system, which intimately bathes and permeates brain and spinal cord tissues, constitutes a circulatory system within the body. Although it has some similarities to systemic vascular and lymphatic circulation, its anatomical arrangement differs considerably. Indeed, this system has been named the “third circulation” system. Due to the extensive area of CSF-tissue contact over the cerebral and spinal cord surfaces, in the paravascular Virchow-Robins spaces, and cerebral ventricles, the cerebrospinal fluid system constitutes a vast, complex and intimate avenue for access to central nervous tissue.

Disclosed herein are methods of treating critical diseases and conditions of neuronal tissue using a perfusion of that tissue, methods of washing out toxic metabolic by-products with appropriate fluids, methods of treating certain neurologic diseases and conditions, and methods of delivering certain classes of agents.

### SUMMARY OF THE INVENTION

In one embodiment, the invention provides a method of treating in an animal that has suffered damage to cerebrospinal tissue or that has an indication creating a risk of damage to cerebrospinal tissue, the method comprising: a. injecting a physiologically acceptable cerebrospinal perfusion fluid into a first catheter into the cerebrospinal pathway, which cerebrospinal perfusion fluid has a neuroprotecting effective amount of a neuroprotectant; b. withdrawing fluid at a second catheter into the cerebrospinal pathway to create a flow and flow pathway between the first and second catheters; and c. maintaining the flow for a period of time adapted to perfuse an affected tissue.

The invention additionally provides a method of treating in an animal that has suffered damage to cerebrospinal tissue or that has an indication creating a risk of damage to cerebrospinal tissue comprising: injecting a cerebrospinal perfusion fluid into a first catheter into the cerebrospinal pathway, which fluid has a neuroprotective effective amount of a neuroprotectant, wherein the cerebrospinal perfusion fluid further comprises one or both of: (i) an emulsion-forming effective amount of a lipid composition comprised of lipids found in biological membranes, or (ii) 0.05-2.0 g/dL albumin; withdrawing fluid at a second catheter into the cerebrospinal pathway to create a flow and flow pathway between the first and second catheters; and maintaining the flow for a period of time adapted to perfuse an affected tissue.

The invention also provides a method of treating a neurodegenerative disease comprising: injecting physiologically acceptable cerebrospinal perfusion fluid into a first catheter into the cerebrospinal pathway, which fluid has a neuroprotective effective amount of a neuroprotectant; withdrawing fluid at a second catheter into the cerebrospinal pathway to create a flow and flow pathway between the first and second catheters; and maintaining flow for a period of time adapted to perfuse an affected tissue.

The invention further provides a method of treating stroke or trauma to cerebrospinal tissue comprising: a. injecting a physiologically acceptable cerebrospinal perfusion fluid into a first catheter into the cerebrospinal pathway, which fluid has a neuroprotective effective amount of a neuroprotectant; b. withdrawing fluid at a second catheter into the cerebrospinal pathway to create a flow and flow pathway between the first and second catheters; and c. maintaining the flow for a period of time adapted to perfuse an affected tissue.

Additionally, the invention provides a method of treating in an animal that has suffered damage to cerebrospinal tissue or that has an indication creating a risk of damage to cerebrospinal tissue comprising: a. injecting a cerebrospinal perfusion fluid into a first catheter into the cerebrospinal pathway, which fluid has a neuroprotective effective amount of a neuroprotectant; b. withdrawing fluid at a second catheter into the cerebrospinal pathway to create a flow and flow pathway between the first and second catheters; and c. maintaining the flow for a period of time adapted to perfuse an affected tissue.

### BRIEF DESCRIPTION OF THE DRAWINGS

FIG. 1 illustrates a perfusion pathway.

### DETAILED DESCRIPTION OF THE INVENTION

In addition to providing neuroprotectants into cerebrospinal passageways, the inventive method can also be used to remove neurotoxic by-products while optionally providing oxygen, glucose, electrolytes and essential amino acids into neural tissue. If used in a rapidly exchanging cerebrospinal fluid perfusion system, such as is described in WO 01/39819 (the perfusion systems described therein are incorporated by reference, see below), the inventive composition and methods can be used both to supply these nutrients and, at the same time, remove metabolic waste.

Diseases and Conditions

Neurological tissue is placed at risk of death or damage under a variety of conditions such as ischemia, hemorrhage, trauma, or exposure to toxic chemicals. In the case of a number of indications such as stroke (including ischemic and hemorrhagic stroke), traumatic brain injury (TBI), brain hemorrhage, spinal cord traumatic injury, ischemia caused by surgical intervention, central nervous system ischemic injury or exposure to toxic chemicals (such as the chemical warfare nerve agents VX (methylphosphonothiotic acid) and Sarin (methylphosphonofluoridic acid)) it would be useful to protect neural tissue from death or damage. This is done by therapeutically treating the tissue with an effective amount of one or more neuroprotectants depending on the situation. Thus, the invention is used to prevent or ameliorate such damage or death.

The invention can also be used to ameliorate damage due to more chronic neurodegenerative diseases or disorders such as Alzheimer's disease, multiple sclerosis, and amyotrophic lateral sclerosis.

Formulation of a neuroprotectant into an artificial cerebral spinal fluid and perfusion of the central nervous system (CNS) offers significant advantage over current methods for treating these diseases and conditions. Currently neuroprotectant therapy is delivered systemically either orally or intravenously in the hope that therapeutic amounts cross the blood-brain barrier and reach the effected CNS tissues. Systemic administration gives little control as to the amount of neuroprotectant that reaches the effected tissue, or the duration of contact of the tissue with an effective amount of the neuroprotectant. CNS perfusion of the neuroprotectant(s) allows control of both dose and duration of exposure of the agent.

The neuroprotectants are selected from the general classes such as excitatory amino acid inhibitors (such as NAALADase inhibitors, gamma-aminobutyric acid (GABA) agonists, adenosine receptor modulators, metabotropic receptor (mGluR) antagonists, calcium channel blockers, or glutamate receptor (NMDA, AMPA, or Kainate) antagonists), free radical inhibitors or scavengers (such as nitric oxide synthase (NOS) inhibitors, antioxidants, cholinesterase reactivators (such as pralidoxime), muscarinic or cholinergic receptor antagonists (such as atropine), inhibitors of lactic acid synthesis, cyclooxygenase inhibitors, or lipooxygenase inhibitors), antiinflammatory agents (including inhibitors of leukocyte adhesion molecules (e.g., ICAM-1, E-selectin, ELAM-1, and VCAM-1)), matrix metalloproteinase inhibitors (e.g., tetracycline, doxycycline, minicycline, batimastat and marimastat), inhibitors of proteases linked to apoptosis or excitotoxic neuronal injury and necrosis, (including caspase and calpain inhibitors), where such inhibitors can act on the enzymes or on their synthesis.

Examples of agents that are neuroprotectants include:

| | AGENT | DOSE |
|---|---|---|
| CLASS | (target) | (μg/mL) |
| Presynaptic | (R,S)-alpha-methyl-4-carboxyphenylglycine | 0.1-10 |
| Excitatory Amino | (C+MCPG) (metabotropic receptor mGluR | |
| Acid Inhibitor | antagonist) | |
| | (S)-2-amino-4-phosponobutyrate (L-AP4) | 0.1-10 |
| | (metabotropic receptor mGluR agonist) | |
| | (2S, 3S, 4S)-alpha-carboxypropyl-glycine (CCG1) | 0.1-10 |
| | (1S, 3R)-1-amino cyclopentane-1,3-dicarboxyleic | 0.1-10 |
| | acid (1S,3R-ACPD) | |
| Postsynaptic | Nimodipine (Ca+2 blocker) | 0.01-1 |
| Excitatory Amino | Nicardipine (Ca+2 blocker) | 0.01-1 |
| Acid Inhibitor | Ziconotide (SNX-111) | 0.001-.1 |
| | Dizocilpine (MK801, NMDA antagonist) | 0.001-.1 |
| | Eliprodil (NMDA antagonist) | 0.001-.1 |
| | Cerestat (CNS-1102, NMDA antagonist) | 0.001-.1 |
| | D(−)-amino-5-phosphonopentanoic acid (D-AP5, | 0.001-.1 |
| | NMDA antagonist) | |
| | Selfotel, (cis-4-phosphonomethyl-2-piperidine | 0.001-.1 |
| | carboxylic acid-i -(cis-2-carboxypiperidine- | |
| | 4-yl)-propyl-phosphonic acid, CGS 19755, | |
| | NMDA antagonist) | |
| | (±)-6-(1(2)H-tetrazol-5- | 0.001-.1 |
| | yl)methyldecahydroisoquinoline-3- | |
| | carboxylic acid (LY-233536, NMDA | |
| | antagonist) | |
| | cis-(±)-4-[(2H-tetrazol-5-yl)methyl]piperidine-2- | 0.001-.1 |
| | carboxylic acid (LY-233053, NMDA | |
| | antagonist) | |
| | Memantine (NMDA antagonist) | 0.01-1 |
| | Remacemide (NMDA antagonist) | 0.01-1 |
| | Dexanabinol (NMDA antagonist) | 0.01-1 |
| | Sinnabidiol (HU-211, NMDA antagonist) | 0.01-1 |
| | [2,3-dioxo-7-(1H-imidazol-1-yl)6-nitro-1,2,3,4- | |
| | tetrahydro-1-quinoxal-inyl] acetic acid | |
| | monohydrate (YM872, AMPA antagonist, | |
| | i.e., antagonist of AMPA subtype of | |
| | glutamate receptors) | |
| | 7-chloro-3-methyl-3,4-dihydro-2H-1,2, 4- | 0.01-1 |
| | benzothiadiazine S,S-dioxide (IDRA-21, | |
| | AMPA antagonist) | |
| | GV150525A (glycine antagonist) | |
| | 1-amionocyclopropanecarboxylic acid (ACPC, | 1-100 |
| | glycine antagonist), and its methyl | |
| | (ACPCM) and ethyl (ACPCE) esters | |
| | R(+)-3-amino-1-hydroxypyrrolid-2-one (R(+)-HA- | 0.01-1 |
| | 966, glycine antagonist) | |
| | R-cis-β-methyyl-3-amino-1-hydroxypyrrolid-2-one, | 0.01-1 |
| | (L-687414, glycine antagonist) | |
| | Ifenprodil (polyamine antagonist) | 0.01-1 |
| | NPS 1506 | 0.01-1 |
| | SYM-2206 (AMPA Antagonist 1,2- | 0.01-1 |
| | dihydropthalazine) | |
| | Licositnel (ACEA 1021) | 0.01-1 |
| | Clomethiazole (GABA agonist) | 0.01-1 |
| | MDL-27192 (GABA agonist) | 0.001-0.1 |
| Free Radical | Ceresine (CPC-211, lactic acid inhibitor) | 0.01-1 |
| Inhibitors | Ascorbic Acid | 0.1-10 |
| | Nitroarginine (NOS inhibitor) | 0.01-1 |
| | Lubeluzole (NOS inhibitor) | 0.01-1 |
| | Steroidal Antiinflammatories | 0.001-0.1 |
| | Non-steroidal Antiintlammatories (NSAIDs) | 0.01-10 |
| | alpha-Phenyl-N-t-butyl-nitrone (NXY-059) | 0.01-1 |
| | Metalloporphirins (AEOL 10150 & 10113) | 0.01-10 |
| Protease | L,L isomer of Z-Leu-aminobutyric acid- | 0.01-1 |
| Inhibitors | CONH(CH₂)₂ (AK275, Calpain inhibitor, | |
| | from Alkermes, Cambridge, MA) | |
| | AK295 (Calpain inhibitor, from Alkermes) 0.01-1 | |
| | Z-Leu-aminobutyric acid-CONH(CH₂)₃- | 0.01-1 |
| | morpholine (CX295, from Cortex | |
| | Pharmaceuticals, Inc.) | |
| | N-benzyloxycarbonyl-Val-Phe (MDL 28,170, | 0.01-1 |
| | Calpain inhibitor) | |
| | z-VAD-CHO (Caspase inhibitor, from Alexis Co., | 0.01-1 |
| | San Diego, CA) | |
| | z-DEVD (Caspase inhibitor, from Alexis Co.) | 0.01-1 |
| Neuro Metabolite | Citicoline (cerebral vasodilator) | 0.001-0.1 |
| | TAK-147 (acetyicholinesterase inhibitor) | 0.001-0.1 |
| | Etanercept (tumor necrosis factor p75 Fc fusion | 0.001-0.1 |
| | protein, TNFR:Fc) | |
| | LY-287041/NNC-11-0309 (muscarinic M1 agonist) | 0.1-10 |
| Cholinesterase | Pralidoxime Chloride (2-[(hydroxy-imino)methyl]- | 1-100 |
| Reactivators | 1-methylpyrdinium chloride) | |
| Muscarinic | Atropine (α-(hydroxymethyl)benzene-acetic acid | 1-100 |
| Cholinergic | (3-endo)-8-methyl-8-azabicyclo[3.2.1]oct-3-yl ester | |
| Receptor | | |
| Antagonists | | |
| Matrix | Tetracycline, | 1-100 |
| Metelloproteinase | Doxycycline, | 1-100 |
| Inhibitors | Minicycline, | 1-100 |
| | Batimastat (BB-94), and | 0.001-0.1 |
| | Marimastat (BB-2516) | 0.001-0.1 |

In addition to those discussed above, the diseases, conditions, syndromes or disorders that can be treated with the invention include neurodegenerative diseases (characterized by progressive loss of neural tissue), neurodegenerative disorders (marked by a loss of nerve cells), and those indications discussed in the following paragraphs of this Section.

Demyelination in later life is a feature of many neurologic disorders; it can result from damage to nerves or myelin due to local injury, ischemia, toxic agents, or metabolic disorders. Extensive myelin loss is usually followed by axonal degeneration and often by cell body degeneration, both of which may be irreversible. However, re-myelination occurs in many instances, and repair, regeneration, and complete recovery of neural function can be rapid. Recovery often occurs after the segmental demyelination that characterizes many peripheral neuropathies; this process may account for the exacerbations and remissions of multiple sclerosis (MS). Central demyelination (i.e., of the spinal cord, brain, or optic nerves) is the predominant finding in the primary demyelinating diseases. The most well known demyelinating diseases are MS and amyotrophic lateral sclerosis (ALS).

In MS, plaques of demyelination, with destruction of oligodendroglia and paravascular inflammation, are disseminated throughout the CNS, primarily in the white matter, with a predilection for the lateral and posterior columns (especially in the cervical and dorsal regions), the optic nerves, and paraventricular areas. Tracts in the midbrain, pons, and cerebellum are also affected as is gray matter in the cerebrum and spinal cord. Cell bodies and axons are usually preserved, especially in recent lesions. Later, axons may be destroyed, especially in the long tracts, and a fibrous gliosis makes the tracts appear sclerotic. Acute disseminated encephalomyelitis is characterized by paravascular CNS demyelination, which can occur spontaneously but usually follows a viral infection or viral vaccination. Adrenoleukodystrophy and adrenomyeloneuropathy are rare X-linked recessive metabolic disorders characterized by adrenal gland dysfunction and widespread demyelination of the nervous system. Adrenoleukodystrophy occurs in young boys; adrenomyeloneuropathy, in adolescents. Leber's hereditary optic atrophy and related mitochondrial disorders are characterized primarily by bilateral loss of central vision, usually affecting young men in their late teens or early twenties. HTLV-associated myelopathy, a slowly progressive spinal cord disease associated with infection by the human T-cell lymphotrophic virus, is characterized by spastic weakness of both legs.

Preferred agents for use in demyelination diseases include tumor necrosis factor inhibitors such as TNRF:Fc and Nerve Growth Factors.

Cerebral contusions and lacerations are severe injuries. Depending on severity, they are often accompanied by severe surface wounds and by basilar skull fractures or depression fractures. Acute subdural hematomas (blood between the dura mater and arachnoid, usually from bleeding of the bridging veins) and intracerebral hematomas are common in severe head injury. Along with severe brain edema, they account for most fatalities. Chronic subdural hematomas may not produce symptoms until several weeks after trauma. Although early diagnosis (2 to 4 wk after trauma) may be suggested by delayed neurologic deterioration, later diagnosis can be overlooked because of the time lapse between trauma and the onset of symptoms and signs. Subdural hematomas are more common in alcoholics and patients >50 yr, in whom the head injury may have been relatively trivial, even forgotten. Epidural hematomas (blood between the skull and dura mater) are caused by arterial bleeding, most commonly from damage to the middle meningeal artery.

Preferred agents for treating cerebral contusions and lacerations include free radical inhibitors and pre- and post-synaptic excitatory amino acid inhibitors.

After a spinal injury, neurologic function may be lost briefly due to concussion, more lastingly due to spinal cord compression caused by contusion or hemorrhage, or permanently due to lacerations or transection. With a contusion, rapid edematous swelling of the cord with increased intradural pressure can result in severe dysfunction for several days. Spontaneous improvement can follow, but some residual disability often remains. Hemorrhage in the spinal cord (hematomyelia) is usually confined to the cervical central gray matter, resulting in signs of lower motor neuron damage (muscle weakness and wasting, fasciculations, and diminished tendon reflexes in the arms), which is usually permanent

Spinal compression, particularly acute spinal compression, spinal abscess and hematoma can also be treated with the methods of the invention.

Preferred agents for use in these kinds of trauma or spinal compression include steroidal and non-steroidal antiinflammatory agents.

Acute transverse myelitis is a syndrome in which acute inflammation affects gray and white matter in one or more adjacent thoracic segments. Some cases follow nonspecific viral infection or vaccination, suggesting an immunologic cause; others are associated with vasculitis, use of amphetamines or IV heroin use, Lyme disease, syphilis, tuberculosis, or parasitic or fungal agents. Usually, symptoms include ascending weakness and numbness of the feet and legs and difficulty voiding develop over a few days; they may progress over several more days to become severe, usually with global sensorimotor paraplegia below the lesion, urinary retention, and loss of bowel control. Occasionally, posterior column functions are spared, at least initially. Local back pain, headache, and stiff neck may be present. Preferred agents include immunoglobulins such as IGG.

Dementias associated with damage to cerebrospinal tissue include Alzheimer's disease. This dementia is a progressive, inexorable loss of cognitive function associated with an excessive number of senile plaques in the cerebral cortex and subcortical gray matter, which also contains β-amyloid and neurofibrillary tangles consisting of tau protein. Lewy body dementia may be the second most common dementia after Alzheimer's disease. Lewy bodies are hallmark lesions of degenerating neurons in Parkinson's disease and occur in dementia with or without features of Parkinson's disease. In Lewy body dementia, Lewy bodies may predominate markedly or be intermixed with classic pathologic changes of Alzheimer's disease. Symptoms, signs, and course of Lewy body dementia resemble those of Alzheimer's disease, except hallucinations (mainly visual) are more common and patients appear to have an exquisite sensitivity to antipsychotic-induced extrapyramidal adverse effects. Cerebrovascular disease can destroy enough brain tissue to impair function, leading to dementia. Vascular dementia, which includes impairment due to single, strategically located infarcts or to multiple small infarcts from small or medium-sized vessel disease, is more common in men and generally begins after age 70. It occurs more often in persons who have hypertension and/or diabetes mellitus or who abuse tobacco.

Preferred agents for use in such dementias include albumin and immunoglobulins such as IGG.

The neuroprotectant is selected based on the indication that creates a risk of damage to cerebrospinal tissue. Identification or diagnosis of the indication is conducted by methods known in the art. Similarly, those of skill in the art can identify appropriate neuroprotectants for the indication.

Cerebrospinal perfusion fluid

In one embodiment, the cerebrospinal perfusion fluid (“CSPF”) is an oxygen-carrying nutrient emulsion according to the following:

| | | | Still More |
|---|---|---|---|
| | | More | Preferred |
| | Preferred | Preferred | Range |
| Component | Range | Range | or Amount |
| Oxygen-Carrying Compound, | 5-15 | 9-11 | 9.5-10-5 |
| % v/v | | | |
| Lipid,mg/mL | 8-14 | 10-13 | 11.5 |
| Albumin, g/dL, | 0.05-2.0 | 1.5-1.9 | 1.67 |
| α-Ketoglutaric Acid, μg/mL | 5-40 | 22-28 | 25 |
| Amino Acids, μg/mL | | | |
| L-Isoleucine + L-Leucine | 5-50 | 11-23 | 17.5 |
| L-Valine | 5-50 | 11-22 | 16.6 |
| L-Alanine | 5-50 | 19-38 | 28.6 |
| L-Serine | 5-50 | 16-33 | 24.6 |
| L-Histidine | 2-20 | 7-14 | 10.3 |
| L-Methionine | 0.1-5 | 1.4-2.8 | 2.1 |
| L-Phenylalanine + L-Lysine | 5-50 | 23-47 | 35.3 |
| L-Threonine + L-Arginine | 5-50 | 32-64 | 48.3 |
| L-Tyrosine | 1-20 | 5-11 | 7.9 |
| Na⁺, mM | 135-150 | 137-147 | 147 |
| K⁺, mM | 2.5-4.0 | 2.7-3.9 | 2.9 |
| Cl⁻, mM | 110-135 | 116-135 | 130 |
| Ca⁺², mM | 1.0-1.6 | 1.0-1.5 | 1.15 |
| Mg⁺², mM | 0.8-1.6 | 1.0-1.5 | 1.12 |
| Glucose (dextrose), mg/dL | 10-150 | 30-100 | 94 |

The pH of the emulsion, or vehicle (constituting the above or the like without oxygen-carrying compound), is in the physiological range, such as about pH 7.3. In one embodiment, the amino acids include tryptophan.

The cerebrospinal perfusion fluid is preferably formulated such that it is physiologic and can directly contact tissues of the neuraxis for an extended period of time, from hours to days, without causing side effects. For best performance, it is believed that the artificial cerebrospinal fluid should be appropriately buffered and contains appropriate amounts of amino acids, electrolytes and other compounds helpful to healthy metabolism. Thus, in preferred methods, these components do not need to be supplied through equilibration with other body fluids. Of course, simpler solutions, such as appropriately balanced salts, are used in neurosurgery and are to some degree acceptable. Where the cerebrospinal perfusion fluid is formulated with nutrients, it can be termed “artificial cerebrospinal fluid” or “ACSF.”

In some embodiments, the cerebrospinal perfusion fluid is simplified CSPF is simplified, such as according to the following:

| | | More | Still More |
|---|---|---|---|
| | Preferred | Preferred | Preferred Range |
| Component | Range | Range | or Amount |
| Albumin, g/dL, | 0.05-2.0 | 1.5-1.9 | 1.67 |
| Na⁺, mM | 135-150 | 137-147 | 147 |
| K⁺, mM | 2.5-4.0 | 2.7-3.9 | 2.9 |
| Cl⁻, mM | 110-135 | 116-135 | 130 |
| Ca⁺², mM | 1.0-1.6 | 1.0-1.5 | 1.15 |
| Mg⁺², mM | 0.8-1.6 | 1.0-1.5 | 1.12 |
| Glucose (dextrose), mg/dL | 10-150 | 30-100 | 94 |

For example, the poly-fluorinated, oxygen-carrying compound is omitted. Or, the amino acid nutrient components can be omitted. Ions are maintained to the degree required to avoid damage to cerebrospinal tissue. Appropriate amounts of oncotic agents are preferred. The cerebrospinal perfusion fluid preferably contains albumin component in the amounts recited above.

Generally, tissues and cells will not fare well if exposed to large volumes of non-physiologic ionic solutions. Accordingly, appropriate electrolyte compositions at the tissue level are important when it is considered that the circulatory method of the present invention could dilute electrolytes from the region, to the detriment of cell membrane function. Desirably, sodium, potassium, calcium, magnesium, and chloride ions are carefully balanced in the neuroprotectant formulations of the present invention to create, to the degree possible, normal extra-cellular compositions.

The neuroprotectant formulations of the invention preferably exclude four amino acids, glutathione, cysteine, ornithine and glutamine, from the group of amino acids included in the formulation, and preferably include sodium bicarbonate in an amount sufficient to increase the buffering capacity of the nutrient solution, in order to more closely resemble the cerebrospinal fluid of the subject.

Kits for conveniently and safely generating fluorocarbon nutrient emulsion or a corresponding vehicle lacking poly-fluorinated, oxygen-carrying compound are described for example in U.S. patent application Ser. No. 09/619,414, filed Jul. 19, 2000 (the specific formulations and kits described therein are incorporated by reference as outlined below).

While not wishing to be limited to theory, it is believed that the lipid and albumin component help flush or perfuse from cerebrospinal tissue metabolic by-products and cellular debris that accentuates cell damage. For example, it is believed that these components help flush cytokines and TNFα, which are believed to lead to shock.

Methodology

In accordance with a preferred method of the present invention, the neuroprotectant formulation is circulated through this cerebrospinal fluid route by injecting it into brain vesicles and withdrawing it from the cisterna magna or the spinal subarachnoid space to nourish and to treat central nervous tissues. In other instances the fluid can be injected into the subarachnoid space and withdrawn from another subarachnoid position. In a preferred embodiment, oxygenated neuroprotectant formulation can be circulated to tissues to be treated in amounts sufficient to provide adequate gas exchange.

The cerebrospinal perfusion fluid (for example comprising a neuroprotectant(s) identified above) can be introduced into the subarachnoid spaces through a catheter that transverses the skull or spinal column and the meninges. The delivery point can be the lateral ventricles, subarachnoid space around the brain, cisterna magna or anywhere along the spine. The cerebrospinal perfusion fluid can be withdrawn from the subarachnoid space from any of these locations using a similar catheter. The cerebrospinal perfusion fluid can be returned to the delivery system, reconditioned as necessary to add components that have been consumed or remove undesirable components that have accumulated, and then returned to the subarachnoid space in recirculating fashion. This process can be continued for days if necessary, thereby directly exposing the neuraxis to the agent over an extended period of time.

Where one seeks to flush out toxic metabolic by-products or particles, the cerebrospinal perfusion fluid is preferably not recirculated. For example, in some embodiments, the withdrawn fluid for a first 3-5 CSF volumes is preferably not recirculated by injection at the first catheter.

This method has several advantages over other routes of administration, such as oral or intravenous administration. This invention provides a method of circulating the neuroprotectant throughout the neuraxis, thus exposing nervous system tissue to the agent in a more uniformly and continuously controlled dose than would otherwise be possible. It also provides a method of maintaining the neuroprotectant within a narrow concentration range, avoiding the necessity of high bolus concentrations over time. According to this method, the nervous system tissue can be exposed to the agent for extended period time, such as days, if necessary. Further, this method minimizes the amount of drug necessary to achieve a therapeutic effect.

It is preferable to establish a flow pathway from the entry catheter (e.g., a ventricular catheter into a lateral ventricle of the brain) to an exit point at a different location in the cerebral spinal pathway (e.g., into the intrathecal space of the lumbar (such as L4-L5) region of the spine) without prematurely inserting an cerebrospinal perfusion fluid containing, for example, neuroprotectant, oxygen-carrying compound, other emulsified components, or the like.

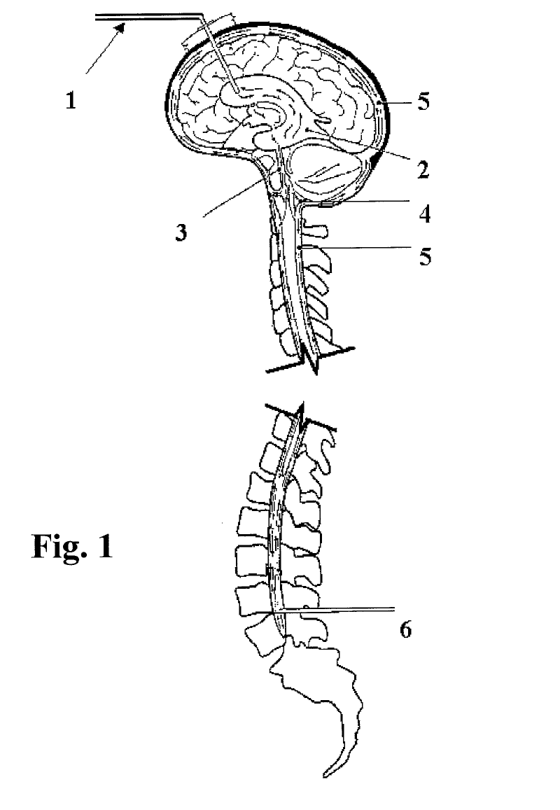

As illustrated in FIG. 1, a ventricular catheter 1 is inserted into a lateral ventrical 2. Via aqueduct 3, cisterna magna 4 and subarachnoid spaces 5, a flow pathway can be established to a lumbar outflow catheter 6. When the inflow and outflow catheters are established (typically with suitable controls to monitor intracranial and intraspinal pressure), vehicle can be used to establish the existence of a flow pathway (such as that illustrated) from the inflow catheter to the outflow catheter. Preferably, the vehicle is infused under gravity feed, with the pressure head designed to avoid excessive intracranial pressure. Once established, the vehicle can be substituted with the cerebrospinal perfusion fluid.

It will be apparent that more than two catheters can be used, though additional catheters are not particularly preferred. Care is taken to monitor the intracranial pressure to assure that flow rates do not cause excessive pressure.

Cerebrospinal perfusion fluid is preferably perfused through the cerebrospinal pathway for a period of time or for a flush volume adapted to effectively reduce the concentration of toxic by-products, or other molecular or debris components resulting from neurological damage. The volume perfused can be for example about 15 CSF volumes, where a “CSF volume” is the average volume of CSF fluid found in animals of comparable age to the subject. Preferably, at least about 1, 2, 4, 8, 15 or 30 CSF volumes are used. In adult humans, for example, a flow rate in the range of 300-3,600 mL/hr is expected, resulting in the exchange of about 2-22 CSF volumes/hr. In human adults, the perfusion is preferably with 300 to 3,600 mL/hr.

Where one seeks to perfuse sufficiently to remove a toxin, perfusion can be maintained for an amount of time or volume effective to diminish toxin concentration at least 10-fold. It will be recognized that the initial “perfusate” against which the reduction is measured is the initial CSF, or that portion of the initial CSF having the highest concentration of toxin.

The perfusion can be conducted, for example, for 6, 12, 24 or 48 or more hours. Preferably the perfusion is conducted for between 6 hours and 48 hours or between 12 hours and 24 hours. More preferably, the perfusion is conducted for at least about 24 hours. Preferably, the perfusion is conducted for no more than about 120 hours (and in one embodiment, no more than about 72 hours).

In one embodiment, the perfusion is an adjunct to a longer term therapy that primarily seeks to deliver the neuroprotectant, and does not seek to perfusion out the cerebrospinal tissue. For example, at some point during the course of the condition the perfusion is conducted to reduce the quantity of neurotoxic by-products. Thereafter, the focus is typically on delivery of the neuroprotectant, rather than perfusing fluid and delivering neuroprotectant. It will be recognized that where the catheters are left in place, these can be used to deliver neuroprotectant by injecting for example 0.1, 0.2, 0.4, 1 or 2 or 5 CSF volumes or less. In some cases, the longer-term delivery will be by a less invasive method, such as intravenous, oral, or any other recognized method of delivery. The post-perfusion administrations are typically done at least daily for at least seven days. If additional perfusions are conducted in this embodiment, the number of perfusions is typically small, such as two or three in total (including the original perfusion).

For chronic conditions, such as neurodegenerative diseases or disorders, it is likely that multiple treatments will be needed. To accommodate this, the perfusion catheters will remain in place, with the ends attached to a multiple access catheter port implanted under the skin. A patient could then receive repeated treatment on a weekly or monthly basis without the risk of repeated surgeries.

Preferred treatment subjects among animals are mammals, preferably humans.

Oxygen-Carrying Compounds

Generally, the preferred compounds for use as non-aqueous oxygen transfer components are fluorocarbons, such as perfluorocarbons, perfluorinated alkyl polyethers, fluoroethers, fluoramines, etc. While compounds within these groups range in molecular weight from 250 to 7000, their selection for use as non-aqueous transport components are based upon the combination of features of the proper vapor pressure, molecular weight, viscosity, ability to form an emulsion, emulsion stability and tissue distribution. Not only do fluorocarbons possess appropriate properties but they are for the most part non-toxic. One chief advantage of the CSF circulation route is that most or all of the neuroprotectant formulation can be removed by washing at the time of treatment termination. In this way long term cellular retention of oxygenating liquids can be avoided.

Poly-fluorinated, oxygen-carrying compounds are known in the art. The basic requirement is effectiveness in carrying physiologically useful amounts of oxygen. Factors involved in selecting preferred such compounds include oxygen capacity, tissue retention (preferably minimized), emulsion stability, toxicity, and the like. Such compounds are described, for example, in: Riess et al., “Design Synthesis and Evaluation of Fluorocarbons and Surfactants for In vivo Applications New Perfluoroalkylated Polyhydroxylated Surfactants”, Biomat. Artif. Cells Artif. Organs, 16:421-430 (1988); Riess, Reassessment of criteria for the Selection of Perfluorochemicals for Second-Generation Blood Substitutes: Analysis of Structure/Property Relationships, Artificial Organs 8:44-56 (1984); Riess, et al., Design, Synthesis and Evaluation of Fluorocarbons and Surfactants for In Vivo Applications New Perfluoroalkylated Polyhydroxylated Surfactants, Biomat. Artif. Cells Artif. Organs 16:421-430 (1988); Riess, et al., Solubility and Transport Phenomena in Perfluorochemicals Relevant to Blood Substitution and Other Biomedical Applications, Pure & Applied Chem., 54:2383-2406 (1982); Yamanouchi, et al., Quantitative Structure-In Vivo Half-Life Relationships of Perfluorochemicals for Use as Oxygen Transporters, Chem., Pharm. Bull., 33:1221-1231 (1985); Lowe, et al., Perfluorochemicals: Blood Substitutes and Beyond Adv. Mater, 3:87-93 (February 1991); Riess, et al., Fluorocarbon-Based In Vivo Oxygen Transport and Delivery Systems Vox Sang, 61:225-239 (December 1991); and Weers, et al., U.S. Pat. No. 5,914,352.

Among preferred poly-fluorinated, oxygen-carrying compounds are those of the formula

CₘFₘ₊₁—CH═CH—CₙFₙ₊₁,

where m and n or independently at least 1 and m+n equals 6 to 10. Preferably, the double bond is trans. One preferred poly-fluorinated, oxygen-carrying compound is trans-Bis-perfluorobutyl ethylene (m and n each equal 4), which is also known as F44E. F44E formulations have a 25% greater oxygen carrying capacity than that of a prior nutrient solution made with perfluorodecalin, Bell et al., Neurology 37: 133, 1987. Formulations comprising F44E are less viscous and relatively easier to perfuse.

Also preferred are those of the formula

CₘFₘ₊₁—O—CₙFₙ₊₁,

where m and n or independently at least 1 and m+n are equals 6 to 9 (or 8). One of the perfluoro alkyls can be substituted with a halo from Br (preferably), Cl or I. Further preferred are those of the formula

CₘFₘ₊₁—R,

where m is 8 (or 10) to 12 and R is Br, Cl, I, or C₁-C₃ alkyl.

Besides fluorocarbon based products, cell-free hemoglobin and liposome encapsulated hemoglobin may also be used as artificial oxygen carriers. Hemoglobin is a 4 subunit protein that is the naturally occurring oxygen carrier in red blood cells. Cell-free hemoglobin rapidly dissociates in the bloodstream, so artificial hemoglobins are chemically modified to prevent breakdown. Artificial hemoglobin's can be the product of surface modification, cross linkage, or polymerization. The production and use of cell-free hemoglobin is detailed in U.S. Pat. Nos. 5,438,041; 5,770,727; 5,952,470; 5,691,453; 5,618,919; 5,599,907; 5,739,011; 5,563,254; 5,449,759; 5,128,452; 5,827,693, and 5,312,808. Hemoglobin can also be prevented from degradation by being encapsulated within a protective barrier, as in the case with liposome encapsulated hemoglobin, the production and use of which is presented in U.S. Pat. Nos. 5,049,391; 4,133,874; 4,776,991; 4,425,334, and 4,532,130.

Especially for certain conditions for which administration of a neuroprotectant is useful, administration in an oxygen-carrying nutrient emulsion is also useful. Conditions for which administration in an oxygen-carrying nutrient emulsion is particularly useful include those which are accompanied by vasospasm resulting in ischemia, such as subarachnoid hemorrhage.

Definitions

The following terms shall have, for the purposes of this application, the respective meanings set forth below.

bioactive agent. A bioactive agent is a substance such as a chemical that can act on a cell, virus, tissue, organ or organism, including but not limited to insecticides or drugs (i.e., pharmaceuticals) to create a change in the functioning of the cell, virus, organ or organism. In preferred embodiments of the invention, methods of identifying bioactive agents of the invention are applied to organic molecules having molecular weight of about 1500 or less.

cerebrospinal tissue. Cerebrospinal tissue includes all tissues bathed by cerebrospinal fluid.

effective amount: The meaning of “effective amount” will be recognized by clinicians but includes an amount effective to reduce, ameliorate or eliminate one or more symptoms of the disease sought to be treated or the condition sought to be avoided or treated, or to otherwise produce a clinically recognizable change in the pathology of the disease or condition. One measure in the present context is a decrease in infarct volume, which can be measured for example with diffusion-weighted magnetic resonance imaging. See, Warach et al., Annals of Neurology 48: 713-722, 2000. Other measures include testing for motor control, coordination, memory, muscle strength, cognition, tactile sensation, vision, hearing, speech, and other measures of the function of cerebrospinal tissue. Those of ordinary skill will recognize that the amount is adjusted for the more pharmacokinetics operative with the invention.

neuroprotectant. Neuroprotectants include bioactive agents that prevent or ameliorate damage to the brain or spinal cord from ischemia, stroke, convulsions, or trauma, or other condition of the cerebrospinal tissue. Neuroprotectants include neuroregenerative agents that have the functional effect of neuroprotection. Some such agents must be administered before the event, but others may be effective for some time after. Such agents act by a variety of mechanisms, but often directly or indirectly minimize the damage produced by endogenous excitatory amino acids.

nutrient-providing effective amount. A nutrient-providing effective amount of a substance is an amount that can be expected, provided sufficient amounts of other nutrients, to increase metabolism or reproduction of mammalian cells compared with nutrient solutions lacking that substance.

oncotic agent. By oncotic agent is meant substances, generally macromolecules, that are of a size that is not readily able to leave the body cavity or other fluid containing body spaces (such as the cerebrospinal pathway, including the cerebral ventricles and subarachnoid spaces) into which they are inserted. Such oncotic agents are exemplified by blood plasma expanders which are known in general as macromolecules having a size sufficient to inhibit their escape from the blood plasma through the circulatory capillary bed into the interstitial spaces of the body. Serum albumin, preferably human serum albumin, is one well known blood plasma protein that can be used as an oncotic agent. Polysaccharide blood plasma expanders are often glucan polymers. For example, Hetastarch (a product of American Home Products) is an artificial colloid derived from a waxy starch composed almost entirely of amylopectin with hydroxyethyl ether groups introduced into the alpha (1-4) linked glucose units. The colloid properties of a 6% solution (wt/wt) of hetastarch approximate those of human serum albumin. Other polysaccharide derivatives may be suitable as oncotic agents in the blood substitute according to the invention. Among such other polysaccharide derivatives are hydroxymethyl alpha (1-4) or (1-6) polymers and cyclodextins. In general, it is preferred that the polysaccharide is one that is non-antigenic. High molecular weight agents such as Dextran 70 having a molecular weight of about 70,000 Daltons are generally less preferred because they increase viscosity of the colloidal solution and impair the achievement of high flow rates. Preferably, the oncotic agent is in an amount effective to provide, in conjunction with other components of a fluorocarbon nutrient emulsion or a nutrient solution, an oncotic pressure of one to seven torr.

respiration. Respiration is the physical and chemical processes by which an organism supplies its cells and tissues with the oxygen needed for metabolism and, preferably, relieves them of the carbon dioxide formed in energy-producing reactions.

respiration-supporting amount. A respiration-supporting amount of oxygen is an amount that would, in model experiments, provide a statistically significant reduction in morbidity following a focal ischemic event.

Where noted above, publications and references, including but not limited to patents and patent applications, cited in this specification are herein incorporated by reference in their entirety in the entire portion cited as if each individual publication or reference were specifically and individually indicated to be incorporated by reference herein as being fully set forth. Any patent application to which this application claims priority is also incorporated by reference herein in its entirety in the manner described above for publications and references.

While this invention has been described with an emphasis upon preferred embodiments, it will be obvious to those of ordinary skill in the art that variations in the preferred devices and methods may be used and that it is intended that the invention may be practiced otherwise than as specifically described herein. Accordingly, this invention includes all modifications encompassed within the spirit and scope of the invention as defined by the claims that follow.

What is claimed:

1. A method of treating in an animal that has suffered damage to cerebrospinal tissue or that has an indication creating a risk of damage to cerebrospinal tissue, the method comprising:
a. injecting a physiologically acceptable cerebrospinal perfusion fluid into a first catheter into the cerebrospinal pathway, which cerebrospinal perfusion fluid has a neuroprotecting effective amount of a neuroprotectant;
b. withdrawing fluid at a second catheter into the cerebrospinal pathway to create a flow and flow pathway between the first and second catheters; and
c. maintaining the flow for a period of time adapted to perfuse an affected tissue.

2. The method of claim 1, wherein the method is adapted to perfuse at least 1 CSF volume.

3. The method of claim 1, wherein the method is conducted in humans and the perfusion volume is 300 mL to 3,600 mL/hr.

4. The method of claim 1, wherein the flow is maintained for between 6 hours and 120 hours.

5. The method of claim 1, wherein the withdrawn fluid for a first 3 CSF volumes is not recirculated by injection at the first catheter.

6. The method of claim 1, further comprising:
d. administering to the animal at least daily over the course of at least seven days a neuroprotecting effective amount of a neuroprotectant, with the majority of administrations conducted by a route of administration that does not use the catheters or which creates a flow that perfuses no more than 5 volumes of fluid resident in the cerebrospinal pathway.

7. The method of claim 1, wherein the damage to cerebrospinal tissue is caused by stroke, a neurodegenerative disease or trauma.

8. A method of treating in an animal that has suffered damage to cerebrospinal tissue or that has an indication creating a risk of damage to cerebrospinal tissue comprising:
a. injecting a cerebrospinal perfusion fluid into a first catheter into the cerebrospinal pathway, which fluid has a neuroprotective effective amount of a neuroprotectant, wherein the cerebrospinal perfusion fluid further comprises one or both of:
1. an emulsion-forming effective amount of a lipid composition comprised of lipids found in biological membranes, or
2. 0.05-2.0 g/dL albumin;
b. withdrawing fluid at a second catheter into the cerebrospinal pathway to create a flow and flow pathway between the first and second catheters; and
c. maintaining the flow for a period of time adapted to perfuse an affected tissue.

9. The method of claim 8, wherein the flow is maintained for 6 to 120 hours.

10. The method of claim 8, further comprising:
d. administering to the animal at least daily over the course of at least seven days a neuroprotecting effective amount of a neuroprotectant agent, with the majority of administrations conducted by a route of administration that does not use the catheters or which creates a flow which perfusions no more than 5 volumes of fluid resident in the cerebrospinal pathway.

11. The method of claim 8, wherein the lipids are phospholipids.

12. The method of claim 8, wherein fluid is adapted to not carry a respiration-supporting amount of oxygen.

13. A method of treating a neurodegenerative disease comprising:
a. injecting a physiologically acceptable cerebrospinal perfusion fluid into a first catheter into the cerebrospinal pathway, which fluid has a neuroprotective effective amount of a neuroprotectant;
b. withdrawing fluid at a second catheter into the cerebrospinal pathway to create a flow and flow pathway between the first and second catheters; and
c. maintaining the flow for a period of time adapted to perfuse an affected tissue.

14. The method of claim 13, wherein the disease is Alzheimer's or multiple sclerosis.

15. A method of treating stroke or trauma to cerebrospinal tissue comprising:
a. injecting a physiologically acceptable cerebrospinal perfusion fluid into a first catheter into the cerebrospinal pathway, which fluid has a neuroprotective effective amount of a neuroprotectant;
b. withdrawing fluid at a second catheter into the cerebrospinal pathway to create a flow and flow pathway between the first and second catheters; and
c. maintaining the flow for a period of time adapted to perfuse an affected tissue.

16. A method of treating in an animal that has suffered damage to cerebrospinal tissue or that has an indication creating a risk of damage to cerebrospinal tissue comprising:
a. injecting a cerebrospinal perfusion fluid into a first catheter into the cerebrospinal pathway, which fluid has a neuroprotective effective amount of a neuroprotectant, wherein the neuroprotectant is (R,S)-alpha-methyl-4-carboxyphenylglycine, (S)-2-amino-4-phosponobutyrate, (2S, 3S, 4S)-alpha-carboxypropyl-glycine, (1S, 3R)-1-aminocyclopentane-1,3-dicarboxyleic acid, nimodipine, nicardipine, ziconotide, dizocilpine, eliprodil, cerestat, D(−)-amino-5-phosphonopentanoic acid, selfotel, (±)-6-(1(2)H-tetrazol-5-yl)methyldecahydroisoquinoline-3-carboxylic acid, cis-(±)-4-[(2H-tetrazol-5- yl)methyl]piperidine-2-carboxylic acid, memantine, remacemide, dexanabinol, sinnabidiol, [2,3 -dioxo-7-(1H-imidazol-1-yl)6-nitro-1,2,3,4-tetrahydro-1-quinoxalinyl]acetic acid monohydrate, 7-chloro-3 -methyl-3,4-dihydro-2H-1,2,4-benzothiadiazine S,S-dioxide, GV150525A, 1-aminocyclopropanecarboxylic acid, ACPCM, ACPCE, R(+)-3-amino-1-hydroxypyrrolid-2-one, R-cis-β-methyl-3-amino-1-hydroxypyrrolid-2-one, ifenprodil, NPS-1506, 1,2-dihydophthalazine, licositnel, clomthiazole, MDL-27192, ceresine, ascorbic acid, nitroarginine, lubeluzole, steroidal antiinflammatories, non-steroidal antiinflammatories, alpha-phenyl-n-t-butyl-nitrone, AEOL 10150 or 10113 metalloporphirin, L,L isomer of Z-Leu-aminobutyric acid-CONH(CH₂)₂, AK295, Z-Leu-aminobutyric acid-CONH(CH₂)₃-morpholine, N-benzyloxycarbonyl-Val-Phe, z-VAD-CHO, z-DEVD, citicoline, TAK-147, etanercept, LY-287041, atropine or pralidoxime;
b. withdrawing fluid at a second catheter into the cerebrospinal pathway to create a flow and flow pathway between the first and second catheters; and
c. maintaining the flow for a period of time adapted to perfuse an affected tissue.
